(19) United States
(12) Patent Application Publication
(10) Pub. No.: US 2025/0000655 A1
(43) Pub. Date: Jan. 2, 2025

(54) Penis Implant with Synthetic Corpora Cavernosa and Bactericidal Barrier

(71) Applicant: AMED, Alajuela (CR)

(72) Inventors: Isaías Herrera Jiménez, San Jose (CR); Víctor Manuel Solano Umaña, San Jose (CR)

(21) Appl. No.: 18/883,574

(22) Filed: Sep. 12, 2024

Related U.S. Application Data
(63) Continuation of application No. PCT/ES2023/070144, filed on Mar. 13, 2023.

(30) Foreign Application Priority Data
Mar. 15, 2022 (ES) 202230211

(51) Int. Cl. A61F 2/26

(52) U.S. Cl. CPC A61F 2/26

(57) ABSTRACT

The present invention relates to a penis implant with synthetic corpora cavernosa and bactericidal barrier, the main embodiment of which includes two cylinders, which are implantable in the corpora cavernosa of the penis, a pumping and deflating system (pump plus valve), to transfer a biocompatible fluid thereto which is partially stored in the pump, in a state of flaccidity of the penis, and interconnection conduits. The synthetic corpora cavernosa, with approximately 40-45% of the total volume of irregular spaces, are configured directly interconnected with a central channel, which allows for a quick and efficient actuation of the implant, filling or emptying the cylinders, providing the required pressure for the erection. The elements in direct contact with the patient are manufactured with materials having bactericidal properties and resistance to biofilm formation, these characteristics being provided by including EDTA-stabilised metallic nanoparticles (Ag, Au). A second embodiment is provided with a reservoir. The pumping/deflating system is coupled between the cylinders and the reservoir.

### CROSS-REFERENCE TO RELATED APPLICATIONS

This application is a continuation of and claims priority to PCT Application No. PCT/ES2023/070144, filed Mar. 13, 2023, which, in turn, claims priority to Spanish Application No. P202230211, filed Mar. 15, 2022, the entire contents of each application is incorporated herein by reference.

### BACKGROUND OF THE INVENTION

### Field of the Invention

The present invention is related to the field of medical implants, and, specifically, it refers to a hydraulic penile prosthesis, with a conveniently improved design, to facilitate the pumping of the liquid that causes the rigidity of the main elements of the penis implant (cylinders with synthetic corpora cavernosa).

### Description of Related Art

It is known that erectile dysfunction is an increasingly common problem, affecting approximately 20% of men, a percentage that increases with age, with the incidence exceeding 60 years of age having 40% affected in that age group.

On the market, penile implants present a widely known solution to the problem of erectile dysfunction, with semi-rigid and inflatable implants being the two most relevant types. Within the latter, which are the most commonly used prostheses, are the so-called “two- and three-piece implants.” The latter include a fluid-filled reservoir (usually saline) implanted under the abdominal wall, a pump, with a release valve, placed inside the scrotum, and two inflatable cylinders inside the corpora cavernosa of the penis. To achieve an erection, a liquid must be pumped from a reservoir element, which acts as a reservoir, into the cylinders. Afterwards, the reverse process is necessary, during which the valve is opened to drain the liquid and return it to the aforementioned tank element.

In order to know the state of the art relevant to the invention described here, the OEPM was requested to prepare a Patent Technological Report (hereinafter, “ITP”, N/Ref.: 100633/P8791), and it was issued by the Office on Oct. 4, 2021. In it, the US patent D01: US2019307565 (A1), titled “Inflatable penile prosthesis having a cylinder with a porous portion” is referenced as a most relevant document.

D01 discloses a three-piece penile prosthesis and a method of manufacturing the same, essentially including a pump, a liquid reservoir, or reservoir, and two inflatable cylinders of porous or spongy structure. Speaking in D01, in any case, of a three-element prosthesis, it is noted that being able to avoid the presence of a reservoir, without loss of effectiveness, as in the case of the invention described in the present invention, involves greater simplicity of the implant surgery and makes it less invasive.

Likewise, the cavernous matrix, disclosed in D01, presents essential differences with the elements of the invention proposed here, as will be detailed later, and specifically the existence of a central and differentiated channel in each of the cylinders, what means a more natural and faster erection for the person receiving the implant than those provided by current implants.

On the other hand, the document, also mentioned in the ITP, D05: WO2019216644A1, refers to a medical implant and a method for modifying its surface, which could adsorb gold nanoparticles providing high prosthesis-patient biocompatibility. Although it is true that, among other uses, the implant can be a penile implant, as the ITP points out, the treatment with nanoparticles would be superficial, influencing the “thiolization” method, which is carried out thanks to the sulfhydryl functional group (—SH), only establishing, as disclosed by D05, a few layers of gold nanoparticles adhered to at least part of the surface of the implant body.

### BRIEF DESCRIPTION OF THE INVENTION

The subject matter disclosed herein describes a penile implant with a surface coating of metallic nanoparticles (gold) not included as in the case described by D05, but rather the material with which all the elements are manufactured, in contact with the patient's body, is a polymeric material that already includes, in its composition, nanoparticles, of silver or gold, which are stabilized in a specific way.

In the case of the invention that will be described below, the modification of the implant in question is not carried out, through the “thiolization” of at least a superficial portion of the implant. It is clear that, in the invention presented here, is not intended to form, nor is it formed, a thiol group-exchanging oxygen atoms for sulfur atoms—to modify a surface oxide film of the implant component and the surface of the gold nanoparticle layer.

Given what is stated here, it is very appropriate to implement different strategies that are less invasive for the patient, and that minimize the risk of future infections, compared to those carried out with the so-called “three-piece prostheses” in widespread use today. which, furthermore, are not configured with any specific surface finish, nor with a particular composition of materials that have antiseptic properties, which would substantially reduce the risk of infections derived from the surgical intervention.

All of this would be achieved with the implant object of the present invention, constituted, according to a main embodiment, by two groups of essential elements (pumping system, which includes a pump, and cylinders), of a specific material, and without therefore decreasing the functional effectiveness of the prosthesis, thus increasing overall patient satisfaction. The fluid filling system included in the present invention is more efficient than those existing in the State of the Art, by obtaining a uniform pressure thanks to a central conduit in interconnection with a series of cavities or branches existing in a synthetic corpus cavernosum housed inside each cylinder, which gives a very natural appearance, simulating the physiological functioning of the penis.

The invention, as expressed in the statement of the present specification, refers to a medical implant, specifically, a penile prosthesis, intended to alleviate erectile dysfunction that occurs when, due to age or other health problems, its erection power is diminished. Said penile prosthesis, compared to known conventional penile implants, provides very advantageous functional characteristics, which will be described in detail below, assuming an improvement on the current state of the art.

The penile prosthesis to be implanted in the patient, object of the present invention, in a first and preferred embodiment, although not the only one, would be among those considered “two-component hydraulic prostheses”, including the following elements:
a pair of inflatable cylindrical elements, which would be implanted within the cavernous area of the patient's penis, and
a pumping system, which includes a pump, connected to the cylinders, which will supply a biocompatible fluid (saline solution)—hereinafter, fluid—to them, in order to inflate them.

And all this, without prejudice to the existence of other components, such as the necessary conduits, for interconnection between both elements.

The particularity of the present invention is that the results are equivalent to three-part hydraulic penile prostheses (cylinders, pump and reservoir), with the advantages that it represents at the time of surgical intervention, not having the third element or reservoir. This is achieved essentially with two actions: a) with the presence, in both cylinders, of a synthetic corpus cavernosum configured with irregular cavities and a central channel, a presence that improves and optimizes their filling, providing optimal rigidity of the penis. in an erect state, and, b) with the placement of a pump that, in turn, will act as a reservoir, when the penis is in a flaccid state; that is, when the cylinders are “deflated”.

Both the central conduit and the cavities or irregular spaces are designed to facilitate the pumping of the fluid, filling the irregular, partially empty spaces more quickly and uniformly, to achieve the required pressure in a more efficient way. Likewise, when emptying the cylinders it will behave in a simpler way than if it only had a porous body without a channel. In this case, the entry and exit of the fluid would be conditioned by the size of the cavities of the porous body.

On the other hand, it is necessary to take into account that the temperature and humidity to which the penile implant will be subjected offers the optimal environment for the existence of microorganisms, such as some of the bacteria that cause possible infections in the patient undergoing surgery. Colonization of surgical implants by microorganisms puts their long-term use at risk, which can be a problem for patients undergoing a penile implant. Colonizing organisms often form biofilms and increase their resistance to multiple antibiotics, making treatment difficult.

For this reason, and to alleviate this circumstance, additionally, all the elements of the penile prosthesis, object of the present invention, that are in contact with the patient's body will be made of a material whose composition includes metallic nanoparticles (silver or gold).

Said material is formed on the basis of a polymer matrix, wherein silver or gold nanoparticles are generated “in situ”, thanks to an oxidation-reduction chemical reaction. With this, an antimicrobial effect is achieved that prevents the proliferation of bacteria that cause possible postsurgery infections. In the present invention, the nanoparticles will be stabilized with a substance that increases their antimicrobial effectiveness, such as ethylenediaminetetraacetic acid (hereinafter, EDTA), thus minimizing the possible resistance of organisms to antibiotics.

On the other hand, the material used, which includes nanoparticles, stabilized with EDTA, as part of it, in the same polymer matrix, that is, in the entire volumetric composition of the material, offers better properties of resistance to erosion or wear than in the case of only a surface coating, so the bactericidal effect is longer than the one a surface coating would offer.

In a second embodiment, and, as an exceptional way, in order to cover the needs of any person potentially receiving the implant, and for any size of penis, a third essential element can be included, which would consist of a reservoir, for the storage of a part of said fluid.

These and other advantages and features of the invention will become apparent to those skilled in the art from the detailed description and the accompanying drawings. It should be understood, however, that the detailed description and accompanying drawings, while indicating preferred embodiments of the present invention, are given by way of illustration and not of limitation. Many changes and modifications may be made within the scope of the present invention without departing from the spirit thereof, and the invention includes all such modifications.

### BRIEF DESCRIPTION OF THE DRAWINGS

Various exemplary embodiments of the subject matter disclosed herein are illustrated in the accompanying drawings in which like reference numerals represent like parts throughout, and in which:

FIG. 1 is a plan view of the location and placement, in the patient, of the two cylinders of the penile implant according to one embodiment of the invention;

FIG. 2 is a side view of the location and placement, in the patient, of the two-piece penile implant of FIG. 1 in a flaccid state;

FIG. 3 is a side view of the location and placement, in the patient, of the two-piece penile implant of FIG. 1 in an erect state;

FIG. 4 is a transverse sectional view of a cylinder in the penile implant of FIG. 1, in which one can see the order of magnitude of the percentage of voids, which will represent between 40 and 45% of the total volume;

FIG. 5 is a partial longitudinal sectional view of a cylinder in the penile implant of FIG. 1, in which one can see the order of magnitude of the percentage of voids, which will represent between 40 and 45% of the total volume;

FIG. 6 is a longitudinal section of the cylinder in the penile implant of FIG. 1, including possible extensions;

FIG. 7 is a schematic representation of the pumping system, which includes the pump and its connection with the conduits;

FIG. 8 is a representation of the composition of the material with nanoparticles as part of its composition;

FIG. 9 is a side view of the location and placement, in the patient, of the three-piece penile implant according to another embodiment of the invention in a flaccid state; and

FIG. 10 details a side view of the location and placement, in the patient, of the three-piece penile implant of FIG. 9 in an erect state.

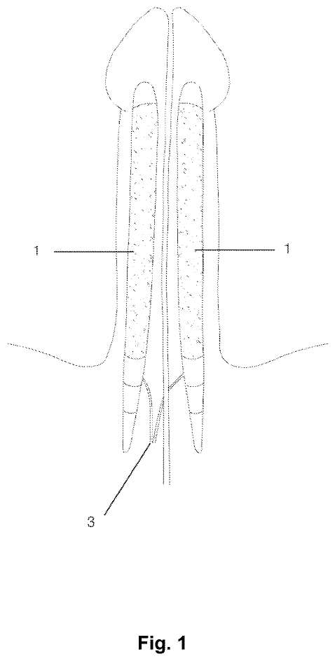

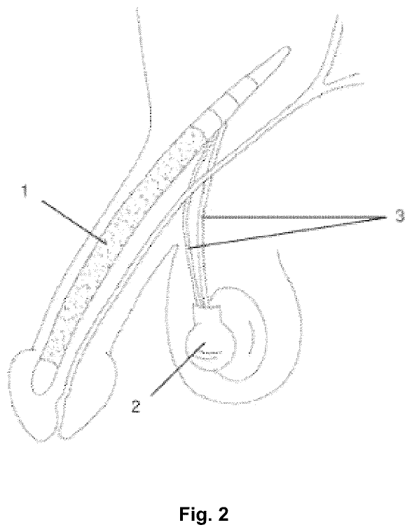

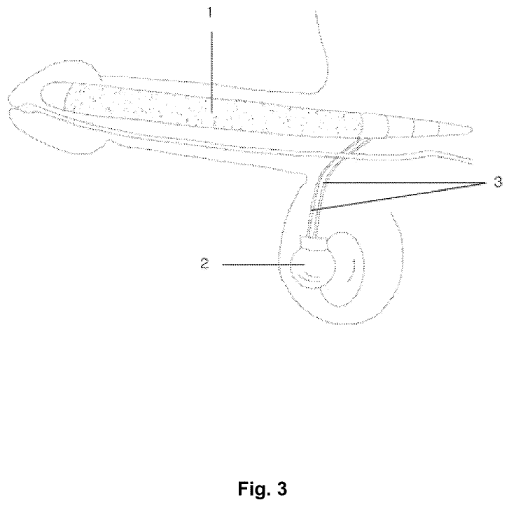

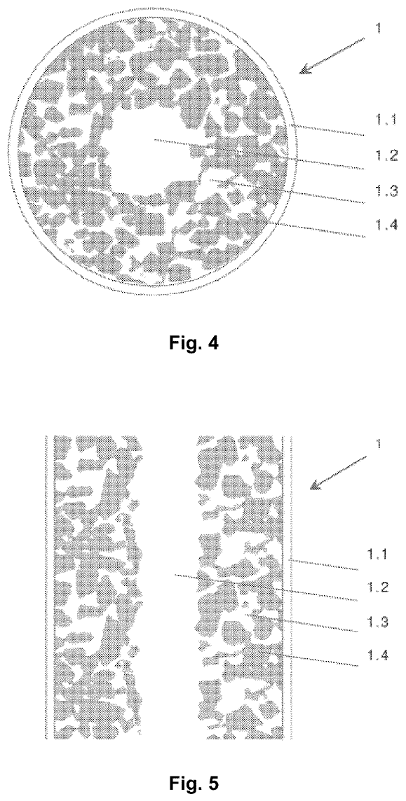

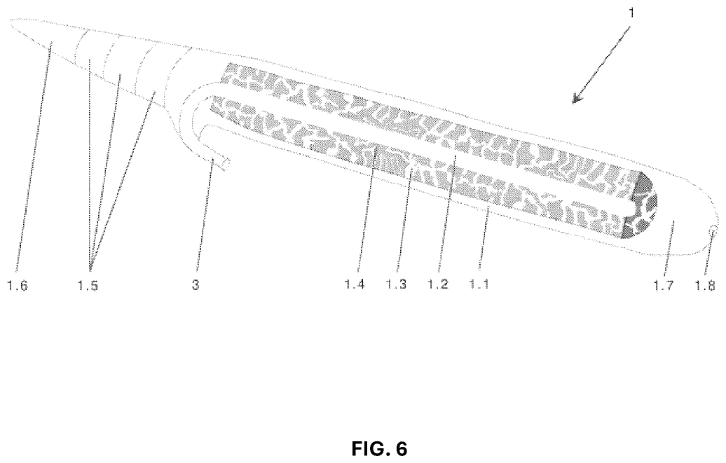

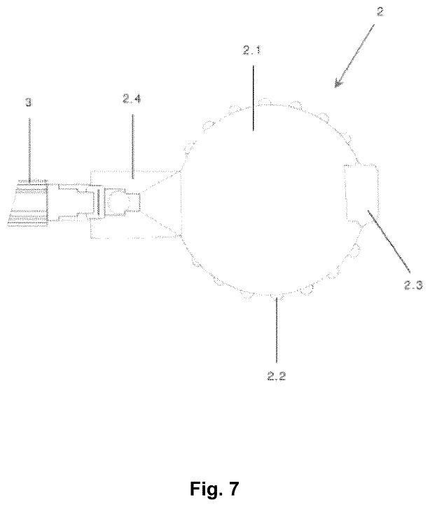

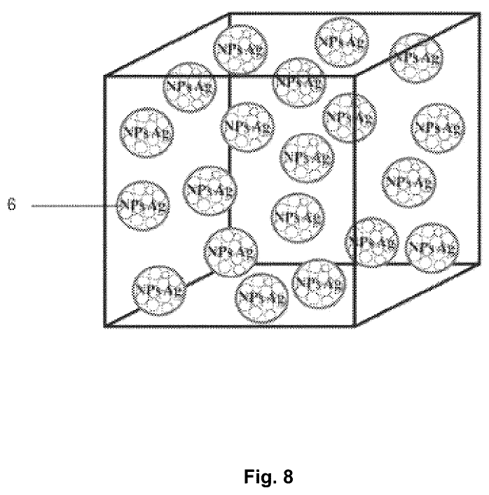

In describing the various embodiments of the invention which are illustrated in the drawings, specific terminology will be resorted to for the sake of clarity. However, it is not intended that the invention be limited to the specific terms so selected and it is understood that each specific term includes all technical equivalents which operate in a similar manner to accomplish a similar purpose. For example, the word “connected,” “attached,” or terms similar thereto are often used. They are not limited to direct connection but include connection through other elements where such connection is recognized as being equivalent by those skilled in the art.

### DETAILED DESCRIPTION OF THE INVENTION

The various features and advantageous details of the subject matter disclosed herein are explained more fully with reference to the non-limiting embodiments described in detail in the following description.

The object of the invention refers to a penile prosthesis, improved with respect to those existing on the market, and, in particular, to a penile implant with synthetic corpora cavernosa, whose irregular gaps or spaces (1.3) represent between 40 and 45% of their total volume, and bactericidal barrier. With this percentage of voids, lightness, rigidity and the formation of a volume necessary to house the necessary amount of liquid are combined, in order to avoid the placement of a reservoir, in the main embodiment, without prejudice to such possibility, in cases of extreme need, such as is indicated in one of the described embodiments.

To first define some concepts and their meaning, in the context of the present invention, the following is indicated:

The use of the term “proximal”, as used herein, refers to a part of an element of the prosthesis, located closest to the center of the patient's body. In the opposite sense, the use of the term “distal” should be understood as referred to the part of said element that is located as far as possible from the point of origin taken as a reference, in this case, the center of the body, of the patient.

In a first, fundamental embodiment, without limitation, the penile implant essentially comprises two cylinders (1) joined to a pump (2.1) through the corresponding valve (2.4) through conduits (3) through which a biocompatible fluid (saline) will circulate. This prosthesis would be included in the so-called “two-piece prostheses”, and therefore, it presents the benefits of those ones, such as having fewer elements that can undergo a possible malfunction (as it does not have a reservoir), and providing a state of flaccidity. greater than the partial flaccidity obtained with a conventional two-piece prosthesis. In addition, it has the advantages of two-piece prostheses, making the surgery less complicated since it does not have a reservoir inserted. It is also noted that, in this main embodiment, since it does not have a reservoir as a third component, the fluid should not be pumped from the reservoir to the pump and from there to the cylinders, this reduces the time to achieve erection by 25 and 50%. On the other hand, since this first embodiment is a “two-piece prosthesis”, there are no connection conduits (3) between a reservoir and the pump (2.1), since there is no reservoir. Therefore, the loss of pressure that would occur due to friction in these hypothetical conduits, of non-negligible length, must not be overcome to achieve erection, thereby reducing the pressure required to mobilize the fluid, between 15 and 20% compared to three-piece prostheses.

The pumping system (2) that includes the pump (2.1) is placed in the scrotum and is easily located by the person receiving the implant thanks to the external protuberances (2.2) that it presents. It acts, at the same time, as a fluid driving system and as a reservoir. The pump (2.1), according to the above, by acting as a kind of reservoir, will contain a large part of saline solution, when the penis is in its flaccid state, and is attached to the cylinders (1) of the implant, through flexible conduits (3), through which the saline liquid will circulate, in a pump-cylinder direction, and vice versa.

The volumetric capacity of both cylinders (1) can vary from 30 to 60 milliliters, depending on their size, according to the needs of each patient. However, the volume of fluid required to fill each cylinder until it reaches its erection is reduced by up to 50%, due to the inclusion, in each cylinder, of a synthetic corpus cavernosum, so the volume necessary to fill both cylinders, when including the synthetic corpora cavernosa (1.4), would be 15 to 30 milliliters. Said volumetric requirement determines the diameter of the pump (2.1), which will be within a range of 3 to 4 centimeters.

In connection with the pump (2.1), there is a valve (2.4) that allows the flow of saline solution towards the synthetic corpora cavernosa and, also, the flow in the reverse direction, back towards the pump (2). When the user presses the pump (2.1), the fluid is transferred to the synthetic corpora cavernosa and the valve (2.4) operates in “non-return” mode. Thus, the fluid is maintained in the corpora cavernosa and the erection occurs; When the person receiving the implant presses the pump (2.1) again, the “non-return” mode is deactivated and the fluid returns, naturally, to the pump (2.1), by suction and gravity, until reaching an equilibrium, and pressing the penis lightly also helps to move the saline solution to the pump (2.1).

The pump (2.1) contains a valve (2.4) that regulates the flow of fluid between the pump (2.1) and the cylinders (1), enabling the flow of fluid in one direction, according to the desired effect. Said valve (2.4) is manipulated, tactilely, by the person receiving the implant, squeezing the actuator (2.3) to open or close. The valve (2.4) serves to allow reverse flow from the cylinders (1) to the pump (2.1) (or, in a three-element embodiment, to the pump (2.1) and the reservoir (4)), when the user selectively activates it.

To start the operation of the prosthesis, the user acts on the pump (2.1), so that the pressure increases and brings the fluid to the cylinders (1). The pumping action causes the two cylinders (1) to expand, thus increasing the length and girth of the penis. When pumping fluid into the cylinders (1), the pressure of the fluid inside the synthetic corpora cavernosa, increases at a level greater than the pressure in the pump (2.1), so that when valve (2.4) is opened, the fluid will have a tendency to flow back to the pump (2.1), until reaching the equilibrium.

The main part of the implant is made up of two synthetic cylinders (1) that will be located inside the natural corpora cavernosa of the penis. Each cylinder (1) has a proximal end (1.6) opposite a distal end (1.7), where, according to the aforementioned interpretation, the distal end (1.7) would be located in the area of the glans, while the proximal end (1.6) would be located in the area of the root of the penis.

Likewise, at least one adapter element (1.5) or “extension” is included, whose function is to adapt the length of the implant to the different physiognomies of the patients, without having to manufacture a high diversity of cylinders to different penis sizes. The number of these adapter elements (1.5) or “extensions” will vary, depending on the patient's needs, so that the proximal end (1.6) of the implant is placed at the base of the pelvic section, the dimensions of the “extensions” can range between 10 and 30 millimeters. The distal end (1.7), commonly called “distal tip”, is a terminal and curved part that has a distal hole (1.8) through which a needle and thread can be passed helping to position the implant within the corpora cavernosa once the surgery is done.

The interior of the cylinders (1) houses two synthetic corpora cavernosa (1.4), which simulate the function of the natural corpora cavernosa of the penis. Both the cylinders (1), in their internal dimensions, and the synthetic corpora cavernosa (1.4) housed inside them, can vary their diameter between 8 and 15 millimeters, and their length between 8 and 20 centimeters, not including the adapter elements (1.5) or “extensions”, nor the distal end (1.7). The wall thickness (1.1) of the cylinders can range from 0.5 to 2 millimeters, depending on the size of the cylinder required by the patient, in each case.

The design of the synthetic corpus cavernosum includes a central channel (1.2) that is responsible for channeling the saline solution to a series of irregular spaces (1.3), which are interconnected with each other, and with the central channel (1.2), of the synthetic corpus cavernosum (1.4). In the present invention, the central channel (1.2) functions analogously to how the central (deep) arteries of the natural corpora cavernosa of the penis would work, and will provide saline solution to the irregular spaces (1.3) interconnected with the central channel (1.2) of the synthetic corpus cavernosum (1.4), with the central channel (1.2) of the synthetic corpus cavernosum (1.4) has a diameter that can vary from 2 to 4 millimeters, to allow greater inflow or outflow. Such dimensional variation corresponds to the variation in diameter that the cylinders (1) can have, depending on the anatomical needs of each patient.

The structure of the synthetic corpus cavernosum (1.4) is, in turn, configured with a series of cavities or irregular spaces (1.3), which represent between 40 and 45% of their total volume, interconnected with each other and with the central channel (1.2), which have a variable diameter, but, in any case, smaller than the diameter of the central channel (1.2). This can be seen in the following two sections, transverse and longitudinal, of the corpora cavernosa.

The central channel (1.2) and the cavities or irregular spaces (1.3), since they interconnected with each other, form multichannels that run the entire length of the synthetic corpus cavernosum (1.4), which helps to fill or to empty the cylinders (1), where the synthetic corpora cavernosa are encapsulated (1.4). The main function of this configuration is to allow the filling or emptying of the synthetic corpora cavernosa (1.4) more efficiently, quickly and with less effort, by the person receiving the implant. This effect is achieved thanks to the significant diameter of the central channel (1.2), which exerts a pressure similar to that exerted by the deep cavernous arteries of the natural corpora cavernosa of the penis, supplying fluid to the interconnected cavities along the synthetic corpora cavernosa (1.4). The almost sudden influx of fluid through the central channel (1.2) would fulfill the function of the blood that causes an erection, by expanding, straightening and hardening the penis.

The synthetic corpora cavernosa (1.4) placed inside the cylinders (1), given their extreme porosity and, essentially, the presence of the central channel (1.2), retain, at all times, a certain volume of fluid, the volume that can be retained can be .25%, or even substantially higher percentages of the total volume of the fluid, what gives the penis a more natural appearance, since the cylinders are not completely emptied, and they retain a quasi-cylindrical shape.

With regard to the materials used in obtaining the penile prosthesis described here, first of all, those elements that are in direct contact with the body of the person receiving it will be distinguished from those that are not, such as the synthetic corpora cavernosa (1.4), which are housed inside the cylinders (1), remaining the cylinder wall (1.1) or the pump (2.1) valve in contact with the patient's body), which will be covered by the casing of the pumping system (2).

Thus, these last elements, and for the reason already stated, as they are not in contact with the patient's body, they do not require specific bactericidal properties, and can be manufactured from various materials. By way of example, and not limitation, synthetic corpora cavernosa (1.4) can be made with poly-dimethyl siloxane (silicone), polyester, polyurethane or cellulose, among other materials; making these elements flexible and elastic. That is, the cylinders (1) and, in particular, the synthetic corpora cavernosa (1.4), are made of a material that provides flexibility when they are deflated and the penis is in a flaccid state, while they expand and stiffen with the entry of fluid, making the penis erection possible.

The rest of the elements, such as the cylinder wall (1.1), the adapter elements (1.5) and the proximal and distal tips (1.6 and 1.7), the pumping system (2) casing and pump-cylinder connection conduits (3) will be made of flexible biocompatible material.

In particular, the connection conduits (3) are made of reinforced silicone, with synthetic fibers in the form of a mesh, with different materials, such as, for example, nylon or polyethylene, among others, which provide greater strength and help to withstand pressure, required in the sexual act, which can reach up to 200 KPa. The cylinders (1) are made up of a wall (1.1), also with multiple layers of flexible silicone (up to 10 layers of material) and, among them, several layers of reinforced silicone (up to three layers), of greater hardness than the previous ones, to reinforce the wall (1.1) and, thus, be in accordance with the pressure requirements necessary for complete sexual functionality. This leads to wall thicknesses (1.1) of the order of those already mentioned (0.5 to 2 millimeters).

The adapter elements (1.5) and proximal and distal ends (1.6 and 1.7) will be made of solid silicone, not containing any internal cavity, except for the distal hole (1.8). The particularity of all types of silicone used in the manufacture of these elements is that they are polymeric compounds, whose composition includes nanoparticles (6) (silver or gold), generated in situ, in the polymer matrix, as shown, schematized in FIG. 8, thanks to a chemical oxidation-reduction reaction. These nanoparticles (6) are stabilized with a substance that enhances the antiseptic properties of the penile implant proposed in the present invention, and, specifically, EDTA, and the different compounds derived from it, such as hydroxy-ethyl-ethylene-diamine-triacetic acid (HEDTA), calcium disodium ethylene-diamino-tetra-acetate (CaNa2EDTA) or other EDTA salts, among others.

The conjunction of the nanoparticles (6) with these substances, for their stabilization, and their inclusion into the constitution of the manufacturing material of most of the components of the penile implant, implies a greater permanence of these nanoparticles (6) in the material of the implant, and, therefore, leads to the achievement of a long-term antimicrobial protection effect, and not in the short term, as it happens in conventional systems, that are not manufactured with such polymeric compounds.

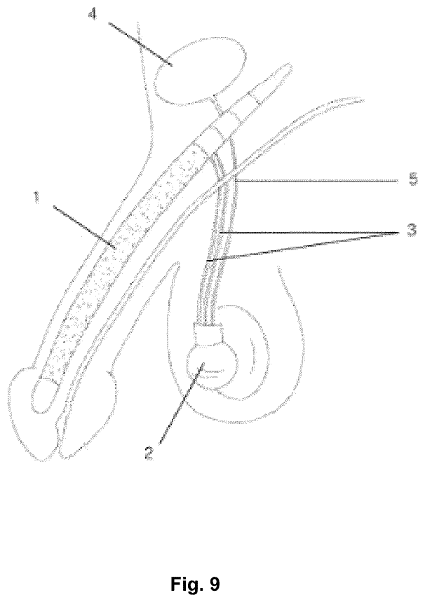

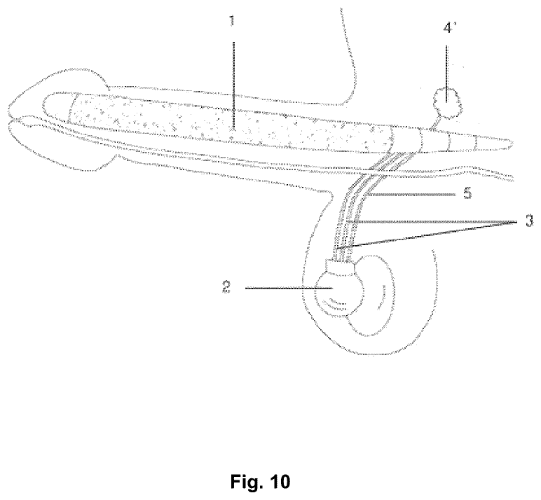

In a second embodiment, illustrated in FIGS. 9 and 10, a penile implant with synthetic corpora cavernosa and a bactericidal barrier is proposed, with three essential elements, adding, to the two-piece prosthesis of the previous embodiment, a reservoir (4) and the pipes (5) necessary to channel the fluid from it to the pump (2.1).

In this case, the proposed system covers those needs of patients who, due to their size, need a greater volume of fluid, which cannot be stored only in the spaces existing in the synthetic corpora cavernosa (1.4) and the pump (2.1). Thus, the excess fluid would be located in a reservoir (4), connected to the pumping system (2), through a pipe (5).

This embodiment offers the advantage that, even requiring the placement of a reservoir (4), its size is reduced by more than fifty percent (50%) compared to a conventional, three-piece implant, which does not contain any cylinders with the technical characteristics of the cylinders (1) specified herein, which facilitates implant surgery and makes it less invasive.

The existence of the synthetic corpora cavernosa (1.4), with their respective central channels (1.2), reduces the amount of fluid that must be pumped into the cylinders (1), which allows the size of the reservoir (4) to be reduced; in both states, erect or flaccid, the penis presents a more natural state that simulates the normal functioning of the system natural penis, and, in addition, the reduction in the volume of saline solution required makes the device more functional in use, since the amount of fluid that moves from one side to the other is smaller.

With regard to the materials used, the information above applies, given that both the reservoir (4) and the pipe (5) are in direct contact with the body of the implant recipient. Therefore, both elements will be manufactured with polymeric compounds already mentioned, whose composition includes the nanoparticles (6) stabilized with EDTA, being, therefore, manufactured with the same material as the conduits (3), the wall (1.1) of the cylinders (1) or the pumping system (2) casing.

Regarding operation, as can be seen in FIGS. 9 and 10, when the penis is in a flaccid state, the reservoir (4) is full of fluid, while, in an erect state, the fluid contained therein, has been pumped to the cylinders (1), leaving the reservoir (4′) slightly minimized in the absence of the pressure exerted by the fluid, which is now in the cylinders (1).

It should be understood that the invention is not limited in its application to the details of construction and arrangements of the components set forth herein. The invention is capable of other embodiments and of being practiced or carried out in various ways. Variations and modifications of the foregoing are within the scope of the present invention. It also being understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

We claim:

1. A penile implant with a synthetic corpora cavernosa and a bactericidal barrier comprising:
two inflatable cylinders, wherein each of the two inflatable cylinders includes an adapter, a proximal end, and a distal end;
a pumping system, further comprising:
a pump, which also acts as a reservoir for part of a fluid inherent to the implant,
a valve,
an outer protective casing, with protuberances, configured to facilitate the user's location, and
an actuator for filling or deflating the cylinders; and
conduits through which the fluid circulates from the pumping system to the cylinders, bidirectionally, depending on the case of filling or deflating the cylinders,
wherein the cylinders include separate synthetic corpora cavernosa, configured with irregular spaces, which represent between 40 and 45% of the total volume of the cylinders, the irregular spaces are connected to each other, and in direct connection with a central channel, constituting, in this way, a system of branches through which the fluid driven from the pump will circulate, the synthetic corpora cavernosa are housed inside the cylinders, being limited by a wall of the cylinders and said synthetic corpora cavernosa include the central channel in the direction of the longitudinal axis of the cylinders.

2. The penile implant with synthetic corpora cavernosa and bactericidal barrier, according to claim 1, wherein the central channel has a diameter of between two to four millimeters.

3. The penile implant with synthetic corpora cavernosa and bactericidal barrier, according to claim 2, wherein the pumping system has a coating, or outer casing, that includes protuberances, and which is made of a polymeric material whose composition includes silver or gold nanoparticles.

4. The penile implant with synthetic corpora cavernosa and bactericidal barrier, according to claim 3, wherein the conduits, the adapters, the proximal end, the distal end, and the wall of the cylinders, are all made of a polymeric material, whose composition includes silver or gold nanoparticles.

5. The penile implant with synthetic corpora cavernosa and bactericidal barrier, according to claim 4, wherein the nanoparticles, of silver or gold are stabilized with a substance with bactericidal effects.

6. The penile implant with synthetic corpora cavernosa and bactericidal barrier, according to claim 5, wherein the substance with bactericidal effects is selected from the group consisting of ethylenediaminetetraacetic acid (EDTA) and its derivatives.

7. The penile implant with synthetic corpora cavernosa and bactericidal barrier, according to claim 5, further comprising:
a reservoir element, configured to contain at least a portion of the fluid, and
a pipe, which connects the pumping system with the reservoir.

8. The penile implant with synthetic corpora cavernosa and bactericidal barrier, according to claim 7, wherein the reservoir, and the pipe, are made of a polymeric material whose composition includes nanoparticles of silver or of gold, which are stabilized with a substance with bactericidal effects, such as ethylenediaminetetraacetic acid (EDTA), or one of its derivatives.

9. The penile implant with synthetic corpora cavernosa and bactericidal barrier, according to claim 8, wherein the substance with bactericidal effects is selected from the group consisting of ethylenediaminetetraacetic acid (EDTA) and its derivatives.
